(19) United States
(12) Patent Application Publication
(10) Pub. No.: US 2023/0380774 A1
(43) Pub. Date: Nov. 30, 2023

(54) Passive Breathing-Rate Determination

(71) Applicant: Samsung Electronics Co., Ltd., Suwon-si (KR)

(72) Inventors: Tousif Ahmed, San Jose, CA (US); Md Mahbubur Rahman, San Jose, CA (US); Yincheng Jin, Port Jefferson, NY (US); Ebrahim Nematihosseinabadi, San Jose, CA (US); Mohsin Ahmed, Sunnyvale, CA (US); Nafiul Rashid, Irvine, CA (US); Jilong Kuang, San Jose, CA (US); Jun Gao, Menlo Park, CA (US)

(21) Appl. No.: 18/198,989

(22) Filed: May 18, 2023

Related U.S. Application Data
(60) Provisional application No. 63/345,314, filed on May 24, 2022.
(60) Provisional application No. 63/444,163, filed on Feb. 8, 2023.

(51) Int. Cl. A61B 5/00; A61B 5/08; A61B 7/00; G16H 40/67; G16H 50/30

(52) U.S. Cl. CPC A61B 5/7278; A61B 5/0816; A61B 5/7221; A61B 5/7285; A61B 5/6803; A61B 7/003; A61B 5/7203; A61B 5/7264; G16H 40/67; G16H 50/30; A61B 5/1118

(57) ABSTRACT

In one embodiment, a method includes accessing, from a first sensor of a wearable device, motion data representing motion of a user and determining, from the motion data, an activity level of the user. The method includes selecting, based on the activity level, an activity-based technique for estimating the breathing rate of the user, which is used to determine a breathing rate of the user. The method further includes determining a quality associated with the breathing rate and comparing the determined quality with a threshold. If the determined quality is not less than the threshold, then the method includes using the determined breathing rate as a final breathing-rate determination for the user. If the determined quality is less than the threshold, then the method includes activating a second sensor of the wearable device and determining, based on data from the second sensor, a breathing rate for the user.

### PRIORITY CLAIM

This application claims the benefit under 35 U.S.C. § 119 of U.S. Provisional Patent Application 63/345,314 filed May 24, 2022, and of U.S. Provisional Patent Application 63/444,163 filed Feb. 8, 2023, each of which are incorporated by reference herein.

### TECHNICAL FIELD

This application generally relates to passively determining a user's breathing rate.

### BACKGROUND

Breathing is fundamental to humans' health and wellness. The breathing rate, which is the number of breaths that a person takes over a period of time (e.g., a minute), is a well-established vital sign related to human health and is often closely associated with a person's respiratory health, stress, and fitness level. In addition, abnormalities in breathing rate can often indicate a medical condition. For example, abnormal breathing rates, which may be breathing rates above 27 breathes per minute (bpm) or breathing rates less than 10 bpm, are associated with pneumonia, cardiac arrest, drug overdose, and respiratory distress.

### BRIEF DESCRIPTION OF THE DRAWINGS

FIG. 1 illustrates an example recorded motion signal and audio signal from a head-mounted device.

FIG. 2 illustrates an example method for monitoring a user's breathing rate.

FIG. 3 illustrates an example implementation of the method of FIG. 2.

FIG. 4 illustrates an example approach for determining an activity level of a user.

FIG. 5 illustrates an example of steps 220 and 225 of the method of FIG. 2.

FIG. 6A and FIG. 6B illustrate example motion data from the process of FIG. 5.

FIG. 7A illustrates an example of steps 220 and 225 of the method of FIG. 2.

FIG. 7B illustrates an example result of the procedure of FIG. 7A.

FIG. 8 illustrates an example of step 245 of the method of FIG. 2.

FIG. 9 illustrates example audio data.

FIG. 10 illustrates an example set of classifications output by a prediction step.

FIG. 11 illustrates an example computing system.

### DESCRIPTION OF EXAMPLE EMBODIMENTS

Many current methods of determining a user's breathing rate are active methods, in that they require some purposeful activity of the user or another person (e.g., a doctor) in order to administer the method. For example, some methods require a user to place a device having a microphone at a specific place on the user's chest or abdomen to capturing breathing motion. However, such methods require active participation by the user, which is inconvenient, and in addition a user's breathing-rate measurements can be affected when a user is conscious of their breathing and/or that their breathing rate is being determined, resulting in data that is not really representative of the user's breathing rate. Passive breathing determinations, in which a user's breathing rate is determined without that user's (or any other person's) conscious awareness or effort, can therefore be more accurate than active determinations. In addition, active determinations can be disruptive, cost-intensive, and labor-intensive, e.g., because a user must stop what they are doing to perform the test or visit a clinical facility in order to have a specialized test performed.

A respiratory belt can perform passive breathing rate determinations, but such belts are often uncomfortable and expensive, as they are uncommon pieces of equipment with a specialized purpose. Some smartwatches attempt to capture breathing rates, for example based on motion sensors or PPG sensors. However, motion data from a smartwatch can be inaccurate for determining a breathing rate, as motion data is discarded when other motion (e.g., arm movement) is present. In practice, a very low percentage (e.g., 3% to 17%) of motion data obtained by smartwatch may actually be retained for breathing-rate determinations. In addition, PPG sensors can be inaccurate in the presence of excessive motion, and individual user differences (e.g., skin tone, wearing habits) can also affect PPG data.

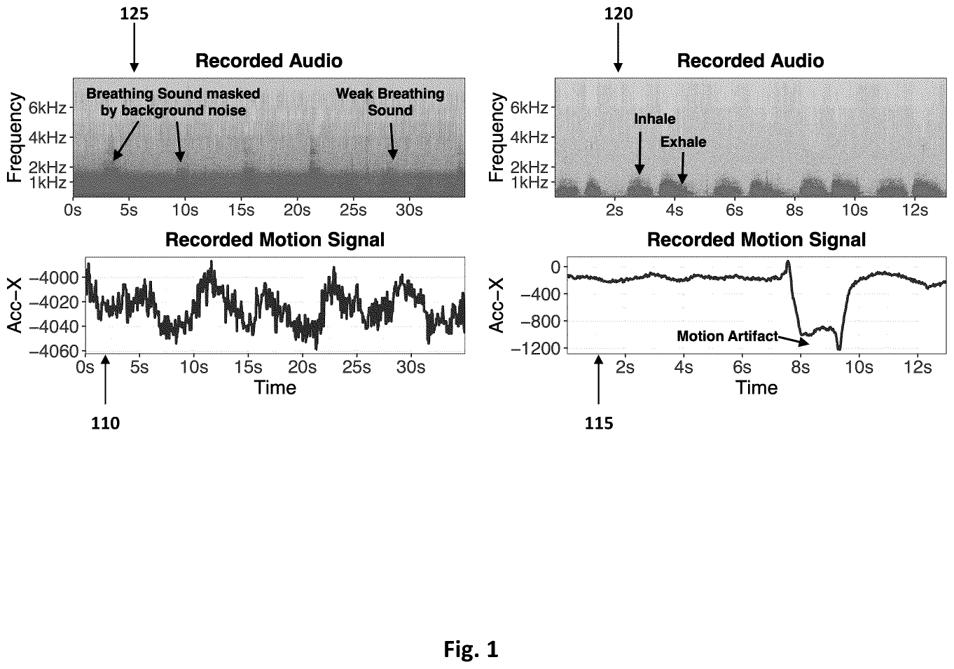

Head-mounted devices, such as earbuds/headphones, glasses, etc., can incorporate multiple types of sensors that can detect data related to breathing rate. For example, a pair of earbuds may have a motion sensor (e.g., an accelerometer and/or gyroscope) and an audio senor (e.g., a microphone) to capture, respectively, head movements related to breathing (e.g., certain vertical head movements that occur when breathing) and breathing sounds generated by the nose and mouth. However, the subtle head motion indicative of breathing can easily be downed out in sensor data when other motion is present. For example, graph 110 in FIG. 1 illustrates an example motion signal recorded by a sensor (e.g., an inertial measurement unit, or IMU) integrated in a head-worn device that captures the periodic motion associated with breathing. In contrast, graph 115 illustrates a motion signal from the same sensor but when other motion is presented. As illustrated in graph 115, the periodic motion signal associated with breathing is difficult to discern due to the presence of other motion that dominates the motion signal. Likewise, breathing-related noises may be drowned out in sensor data by background noises. For example, graph 120 in FIG. 1 illustrates an example recorded audio signal from a sensor integrated in a head-worn device that captures the periodic audio signal associated with breathing. In contrast, graph 125 in FIG. 1 illustrates an audio signal from the same sensor, but background noise is dominating the captured data in graph 125, making it difficult to discern the breathing-related audio data.

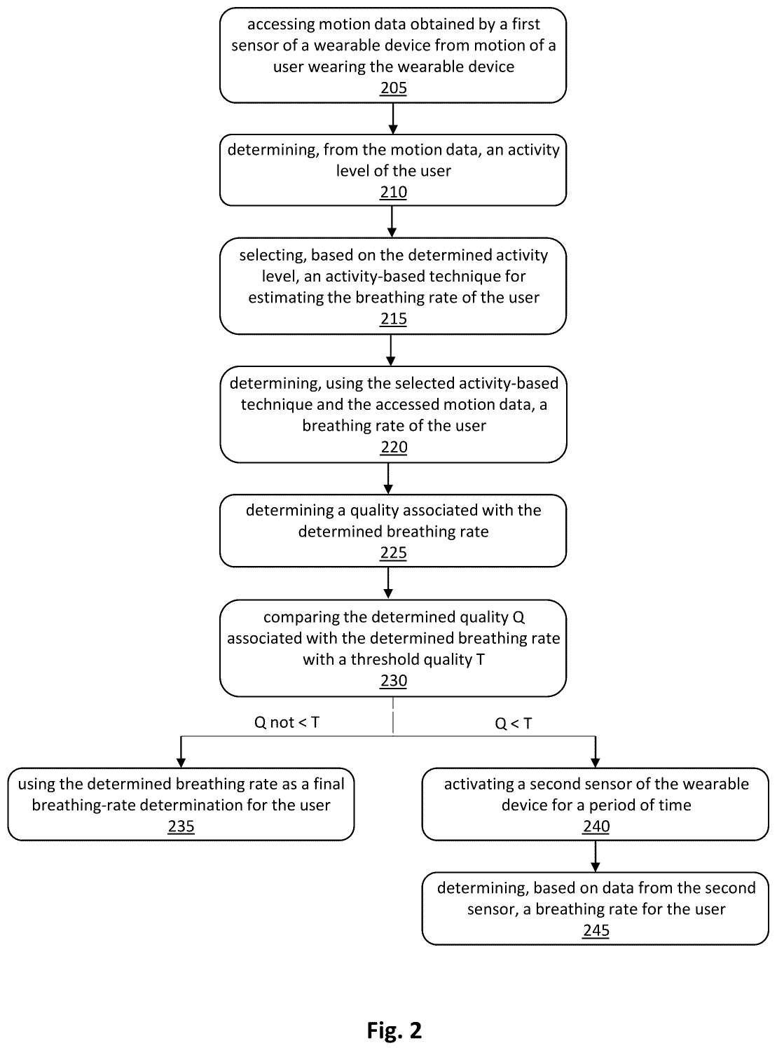

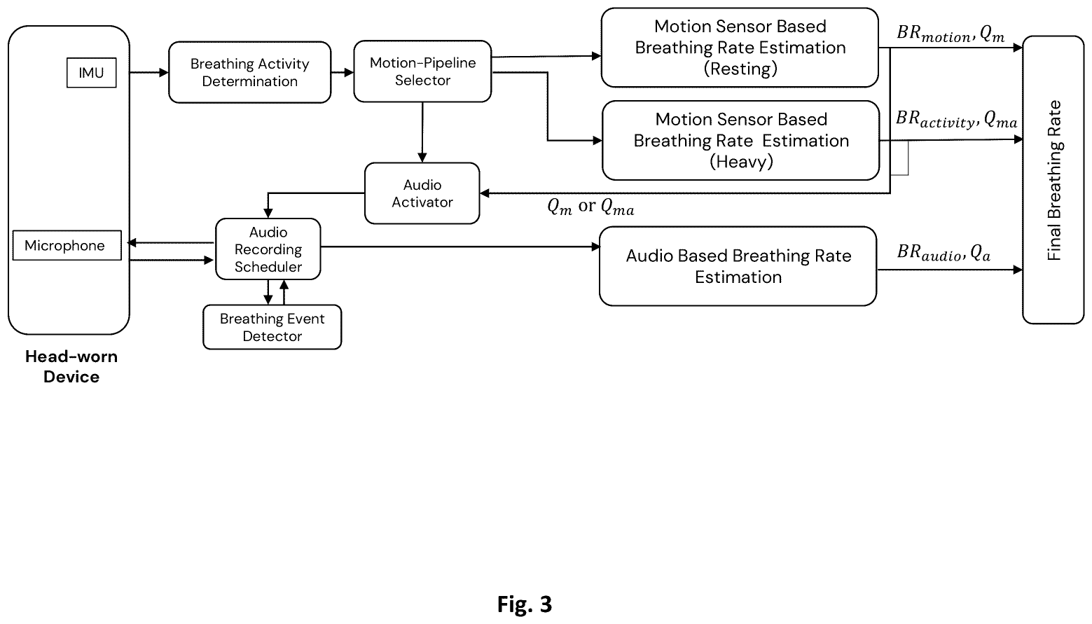

FIG. 2 illustrates an example method for accurately, efficiently, and passively monitoring a user's breathing rate using a default motion sensor and intelligent activation of another sensor, such as an audio sensor. FIG. 3 illustrates an example implementation of the method of FIG. 2 in the context of a head-worn device (such as a pair of earbuds) that includes a six-axis IMU and a microphone.

Step 205 of the example method of FIG. 2 includes accessing motion data obtained by a first sensor of a wearable device from motion of a user wearing the wearable device. For example, the first sensor may be an IMU, and the wearable device may be a pair of earbuds. In particular embodiments, some or all of the steps of the example method of FIG. 2, including step 205, may be performed by the wearable device or by another device, e.g., by a smartphone or server device connected to the wearable device either directly or via an intermediary device.

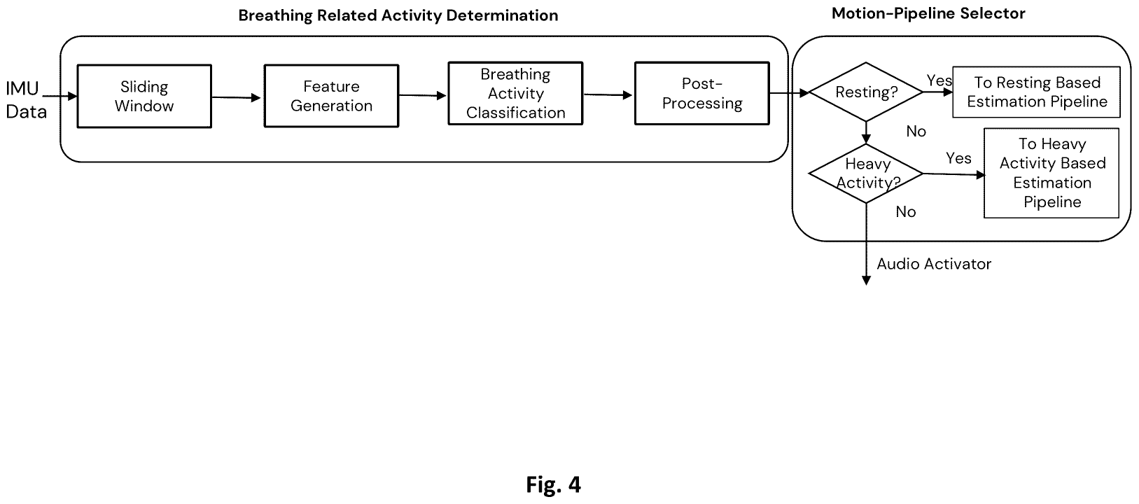

Step 210 of the example method of FIG. 2 includes determining, from the motion data, an activity level of the user. The determined activity level may be selected from a set of predetermined activity levels, such as resting, low activity, medium activity, high activity, etc. FIG. 4 illustrates an example approach for determining an activity level of the user in the context of a predetermined “resting” activity level and a predetermined “heavy” activity level. In particular embodiments, “heavy” activity can include but is not limited to physical activity like High Intensity Interval Training (HITT) such as 3-Minute Step Test and the like, or walking such as 6-Minute Walk Test and the like. In particular embodiments, step 210 may be performed by a machine-learning architecture, such as a lightweight machine learning-based approach, that identifies a user's head motion from the motion-sensor data. In the example of FIG. 4, this can be a three-class classification approach, which predicts whether activity is classified as “resting,” “heavy,” or “other.” The “other” class may represent, among other things, random movements made by a user, and because breathing-related movements are not periodic during many random movements, it is difficult to reliably determine breathing rate from corresponding motion data. For example, during speaking, eating, or drinking breathing happens randomly (i.e., not in the typical periodic pattern a person usually breathes with), and therefore motion signals during these activities are unlikely to produce identifiable breathing data.

In the example of FIG. 4, a sliding window is applied to motion data accessed in step 205. In particular embodiments, steps 205 and 210 may happen substantially continuously and in real time. Therefore, a sliding window may be continuously updated and evaluated as a new motion data segments from the sensor are obtained. In the example of FIG. 4, features are extracted from the raw motion data within a window. In particular embodiments, a few (e.g., 10) statistical features may be extracted, for example in order to keep the approach lightweight. In particular embodiments, the features used for motion activity classification may be identified using a correlation-based feature selection (CFS) algorithm on more than 200 commonly used time-domain features for activity recognition. This disclosure contemplates that any suitable features may be used to classify an activity level from motion data, such as jerking, signal magnitude area, mean magnitude, etc. This disclosure contemplates that any suitable classifier or classifiers may be used to classify activity level based on the features exacted from a window of motion data. For example, random forest and/or logistic regression classifiers may be used, with the latter being particularly suited to lightweight classification, in particular embodiments.

Step 215 of the example method of FIG. 2 includes selecting, based on the determined activity level, an activity-based technique for estimating the breathing rate of the user. For example, as illustrated in the example of FIG. 4, if the user's activity level is determined to be “resting,” then a corresponding resting-based technique (explained more fully below) is used to determine the user's breathing rate. Similarly, if the user's activity level is classified as “heavy,” then a corresponding heavy-activity-based technique (also explained more fully below) is used to determine a user's breathing rate. As explained more fully herein, in particular embodiments, if the determined activity is not associated with any activity-based technique for estimating the breathing rate of the user (e.g., the user's activity is classified as “other”), then a second sensor (e.g., a microphone) may be activated. In particular embodiments, activation of a second sensor may depend on the output of an activation pipeline, as discussed more fully herein.

Step 220 of the example method of FIG. 2 includes determining, using the selected activity-based technique and the accessed motion data, a breathing rate of the user. Step 225 includes determining a quality associated with the determined breathing rate, and step 230 includes comparing the determined quality associated with the determined breathing rate with a threshold quality. As explained more fully herein, the quality is used to determine whether the breathing-rate determination is sufficiently reliable to use as an estimation of the user's actual breathing rate.

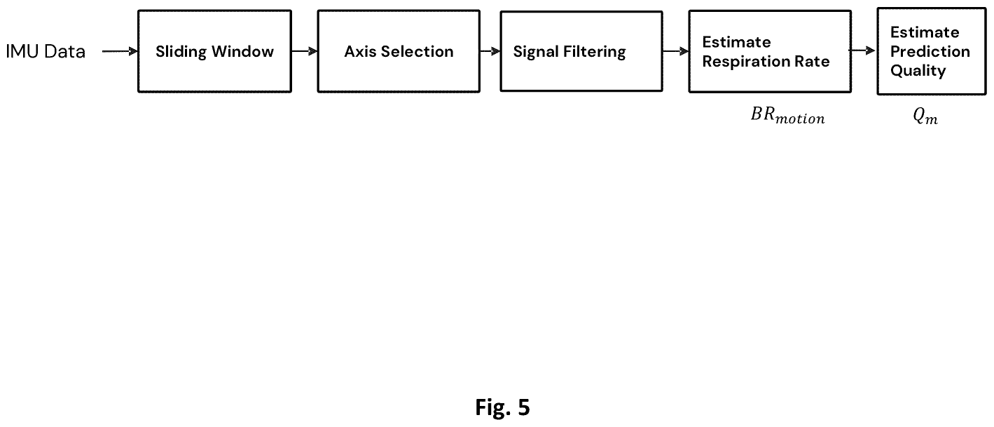

FIG. 5 illustrates an example of steps 220 and 225 in the context of a particular technique associated with a “resting” activity level of a user. The example of FIG. 5 is relatively lightweight, and input data may be, e.g., 3-axis accelerometer or 3-axis gyroscope or full 6-axis data from an IMU. A sliding window is applied to the input IMU data. For example, a 30 second sliding window may be used, which in some embodiments strikes a balance between accuracy and performance demands. Data within the sliding window is divided into a number of steps. For example, a 30 second window may be divided into 2 15-second steps.

The example of FIG. 5 includes selecting an axis from the 6-axis IMU data. Motion data relative to the selected axis will be subsequently used to determine the user's breathing rate (e.g., data with respect to the other 5 axes need not be considered in subsequent steps of the process). In particular embodiments, an axis-selection process selects the axis with the most periodic motion signal, as breathing while resting tends to provide a periodic signal. As an example of periodic axis selection, particular embodiments may perform the following steps on each axis: 1) apply a median filter and then normalize the signal, 2) apply a Butterworth Bandpass filter with, e.g., lowcut and 0.66 Hz highcut frequency, and 3) perform a Fast-Fourier transform (FFT) on the filtered signal. In this example, the axis with a maximum frequency value yields the most periodic axis with the largest signal variation attributable to breathing. Then, subsequent steps in the process use data corresponding to the selected axis for pre-breathing rate estimation. While the example of FIG. 5 selects a single axis from the IMU data, this disclosure contemplates that any suitable number of axes may be selected.

The example of FIG. 5 includes filtering the data about the selected axis. For example, particular embodiments may perform normalization on the filtered signal and apply a 3^{rd} order Butterworth Bandpass filter with 0.13 Hz lowcut and 0.66 Hz highcut frequency. These cutoff frequencies correspond to a breathing rate of between 8 to 40 BPM, but this disclosure contemplates that other cutoffs may be used. Particular embodiments then apply a 2nd order Savitzky-Golay filter for further smoothing the signal.

The example of FIG. 5 includes estimating a breathing rate/respiration rate based on the filtered data signal. In particular embodiments, the filtered signal may resemble a breathing cycle (typically inhalation-pause-exhalation), e.g., due to the step size used. Particular embodiments may use one or more breathing-rate algorithms to arrive at a final breathing-rate determination. For example, particular embodiments may use a Zero-Crossing Based Algorithm (BR_{ZCR}), which identifies the zero-crossing points in the filtered signal and calculates breathing rate based on the time between two zero-crossing points. Particular embodiments may take the median time of the zero-crossing duration to estimate the breathing rate, and the breathing rate can then be calculated as

$$B {R}_{Z C R} = : \frac{60}{median ( zero crossing duration )}$$

Particular embodiments may determine a breathing rate based on an FFT-Based Algorithm (BR_{FFT}), which applies an fast-Fourier transform algorithm to the filtered signal to compute the frequency domain representation of the breathing signal. The breathing rate can then be calculated by selecting the frequency of highest amplitude (peak) within, in the example of FIG. 5, the 0.13 Hz and 0.66 Hz frequency range (7.8 to 40 BPM). In this approach, the rate is calculated as BR_{FFT}=60×argmax FFT(x).

Particular embodiments may determine a breathing rate based on a Peak-Based Algorithm (BRₚₑₐₖ), which uses a peak-detection algorithm to find the peaks and valleys in the filtered signal. Each valley-to-peak indicates an inhale cycle, and the peak-to-valley indicates an exhale cycle. In an ideal breathing cycle, peaks and valleys must occur in an alternating order. However, false peaks or valleys can be detected due to noise in the derived breathing cycle. For that reason, particular embodiments remove the false peaks and valleys when there are multiple peaks in between two valleys or multiple valleys in between two peaks. When there are multiple peaks in between valleys, or vice versa, particular embodiments select the peak that is closest to the nearest valley or the valley that is closest to the nearest peak, respectively. Particular embodiments can then estimate the breathing rate by taking the median peak-to-peak distances. In this approach, the rate is calculated as

$$B {R}_{p e a k} = \frac{60}{median ( peak - to - peak duration )}$$

Particular embodiments may use multiple breathing-rate algorithms to determine a final breathing rate. For example, particular embodiments may determine a breathing rate based on each of BR_{ZCR}, BR_{FFT}, and BRₚₑₐₖ. For example, a final breathing rate may be determined by taking the median of the three estimations for the user's breathing rate. Other approaches may also be used, for example by taking a weighted sum of the separate determinations, etc.

In particular embodiments, a quality determination for a breathing-rate estimation may be based on statistical properties of a set of one or more breathing rate determinations. For example, in the example in which three breathing-rate determinations are used to arrive at a final breathing rate, step 225 of the example method of FIG. 2 can include determining a quality Qₘ by taking the standard deviation of the three determinations, i.e.:

Qₘ=σ(BR_{zcr},BR_{fft},BRₚₑₐₖ)

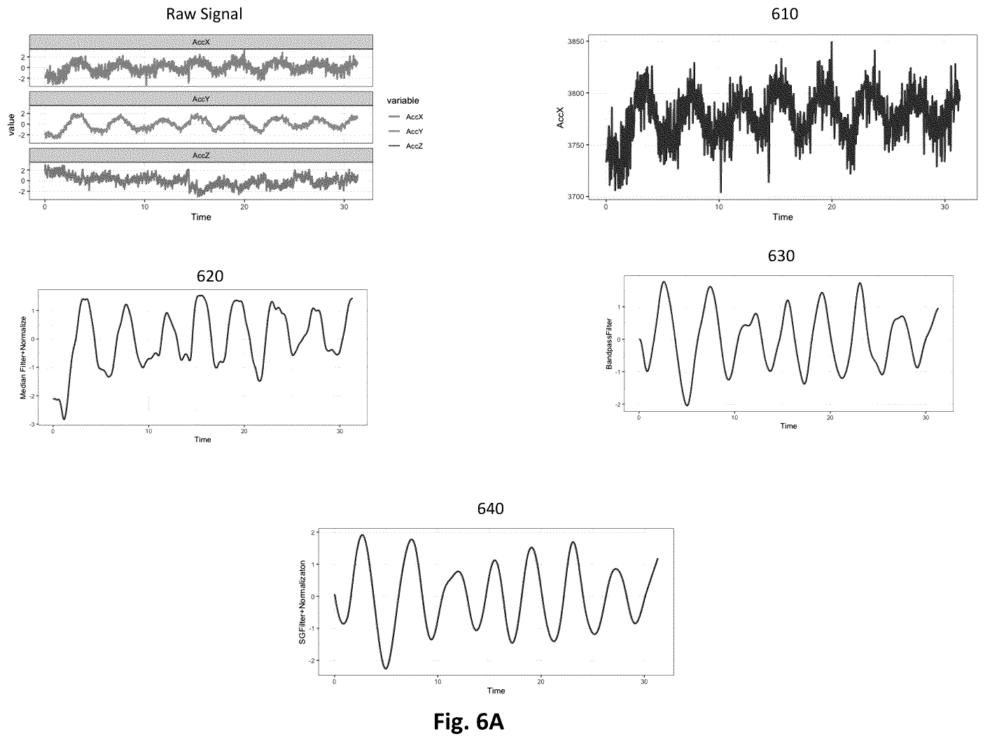

FIG. 6A and FIG. 6B illustrate example data obtained by an example implementation of the example of FIG. 5. As illustrated in FIG. 6A, a raw signal is obtained. The signal about three axis is shown in FIG. 6A. Graph 610 illustrates the result of axis selection, e.g., that the signal about the x axis has been selected for further processing. Graph 620 illustrates the resulting signal after a median filter and normalization is applied. Graph 630 illustrates the resulting signal after bandpass filter, and graph 640 illustrates the resulting signal after a Savitzy-Golay filter is applied. Graph 650 in FIG. 6B illustrates the zero-crossing estimation. Graph 660 illustrates the peak-to-peak estimation method, and graph 670 illustrates the FFT breathing-rate estimation method, after converting the signal (i.e., the signal shown in graph 640) to the frequency domain.

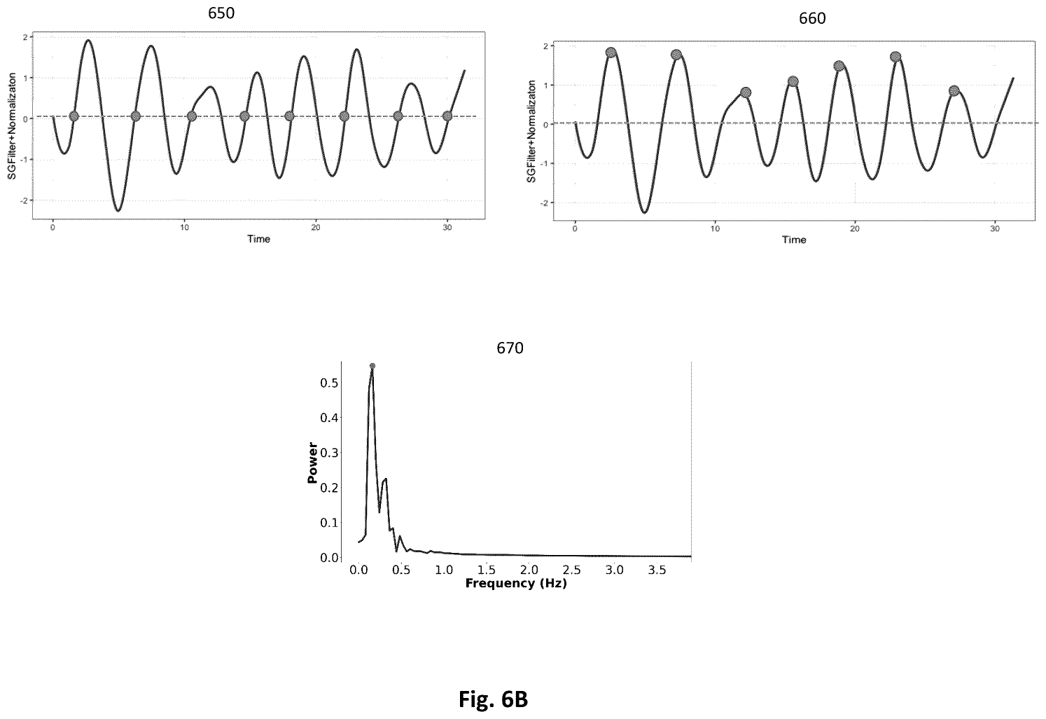

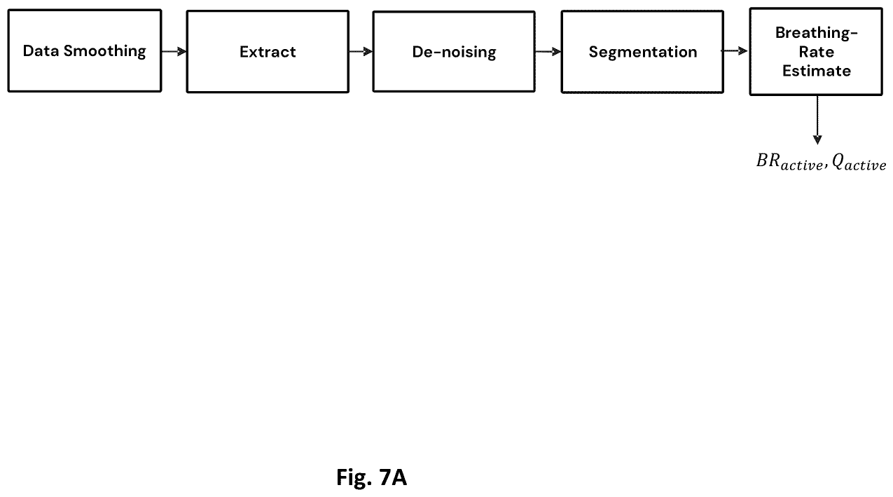

FIG. 7A illustrates an example of steps 220 and 225 in the context of a particular technique associated with an activity level of a user that corresponds to an “active” classification. In particular embodiments, the input data may be a 3-axis IMU signal, such as sensor data from an accelerometer or from a gyroscope, which can reduce computational resources required to estimate a breathing rate relative to using a 6-axis IMU signal. In particular embodiments, a data smoothing step of FIG. 7A can include calculating the square root of x²+y²+z² as breathing-estimation signals where x, y, and z are the three axis signal values from the corresponding IMU sensor. Then, particular embodiments may use a sliding window of, e.g., 4 seconds, in order to smooth out the 3-axis IMU data.

In particular embodiments, a wearable device may be a pair of earbuds, and a user's breathing signals collected from an ear canal may be correlated with small variations in their breathing intervals. Therefore, in order to extract breathing rate, particular embodiments extract these intervals from the comparably large, noisy hearable IMU signals. Particular embodiments may perform this extraction using the following formula:

$${f}^{″} = \frac{( 4 {f}_{0} + {f}_{1} + {f}_{- 1} - 2 ( {f}_{2} + {f}_{- 2} ) - ( {f}_{3} + {f}_{- 3} ) )}{16 {h}^{2}}$$

Here, fᵢ refers to the value of the magnitude time series i samples away, and h is the average time interval between consecutive samples. Then, a differential filter with four steps may be used to obtain a signal that is proportional to acceleration, which is effective for extracting the relatively small motion signals due to breathing. In particular embodiments, noise may be removed from the data in a de-noising step, for example, by applying one or both of outlier detection and a Gaussian filter.

As illustrated in in FIG. 7A, particular embodiments may segment the de-noised data into segments that corresponding to a breathing cycle. For example, particular embodiments may first initialize a template vector as 0, and then search every point in the timeseries to find the most similar segments during the possible range. Particular embodiments may set the batch size, for example to balance between computation cost and accuracy. The similarity between segments can be calculated by dynamic time warping (DTW) distance. For instance, an example approach may take sequence x as an input and output the segmentation by repeating the following:

S^{l+1}=DistDTW(x,μ^{l})

μ^{l+1}=DistDTW(x,S^{l+1})

where S is a set of segments and μ is a template segment. FIG. 7B illustrates an example result of the process of FIG. 7A, which illustrates examples of segments 710 as defined by the vertical bars in FIG. 7B superimposed on a IMU signal, which includes both activity motion and breathing motion.

In particular embodiments, the breathing rate predication of FIG. 7A is calculated by calculating the segments such that BR_{activity}=length(segs), assuming that 60 seconds of data is being analyzed. Since the user will be breathing at a slightly higher rate, we can expect more breathing activity segments, and therefore in particular embodiments Qₐ is set to 0 if length(segs) is less than 1, and Q a is set to 1 otherwise. In this example, a corresponding quality threshold may have a value of 1.

In the example method of FIG. 2, in response to a determination that the determined quality is not less than the threshold quality, then step 235 includes using the determined breathing rate as a final breathing-rate determination for the user. This breathing rate may be stored, e.g., along with a time stamp. In particular embodiments, this breathing rate may be displayed and updated at or near real-time to a user, e.g., on a UI of a health-related application running on the wearable device or on a connected device, such as the user's smartphone. As explained below, this data may be sent to medical personnel, and/or may be used to raise alerts to the user or to medical personnel if a health-related emergency occurs.

In the example method of FIG. 2, in response to a determination that the determined quality is less than the threshold quality, then step 240 includes activating a second sensor of the wearable device for a period of time, and step 245 includes determining, based on data from the second sensor, a breathing rate for the user. As explained herein, in particular embodiments step 240 may occur when no motion-based technique for determining user's breathing rate is detected (e.g., when the user's motion is classified as “other” in the example above).

Before activating the second sensor in step 240, particular embodiments may first use a second-sensor activation process to determine whether to activate the second sensor, even when the quality of a motion-based breathing rate is less than a threshold. For example, an activation process may check whether the duration since the last valid breathing-rate determination is greater than a threshold duration D_{b}. If not, then the process may not activate the second sensor (e.g., step 240 is not performed), and the process may loop back to step 205. If the time since the last valid breathing-rate determination is greater than a threshold, then step 240 may be performed. In particular embodiments, an activation process may check whether the duration since the second sensor was last activated is greater than a threshold duration Dₐ. If not, then the process may not activate the second sensor (e.g., step 240 is not performed), and the process may loop back to step 205. If the time since the second sensor was last activated is greater than a threshold, then step 240 may be performed.

In particular embodiments, an activation process may check whether the duration since the last valid breathing-rate determination is greater than a threshold duration D_{b} and whether the duration since the second sensor was last activated is greater than a threshold duration Dₐ. Step 240 may not be performed unless both checks are satisfied. In particular embodiments, these checks may conserve system resources by not activating the second sensor (e.g., microphone) when the sensor was just recently activated or when valid breathing-rate data (from any source) was recently obtained.

In particular embodiments, the value of D_{b} may vary based on, e.g., a user's preferences and/or on the user's current detected motion. For example, as explained more fully below, a user may indicate how frequently they want a second sensor to activate and/or may indicate a power-setting configuration, and these preferences may adjust D_{b} accordingly. As another example, if a user's motion indicates the user is resting, then D_{b} may be relatively high. On the other hand, if a user's motion indicates that the user is active, or that the user is undergoing unclassified movement (e.g., “other”), then D_{b} may be relatively low. Similarly, the value of Dₐ may vary based on, e.g., a user's preferences and/or on the user's current detected motion. For example, if a user's motion indicates the user is resting, or that the user is undergoing unclassified movement, then Dₐ may be relatively low. On the other hand, if a user's motion indicates that the user is active, then Dₐ may be relatively high.

After step 240 and before step 245, particular embodiments may activate the second sensor and analyze the data to initially determine whether the user's breathing rate can be detected from the data. For example, step 240 may include activating a microphone, e.g., for 10 seconds and particular embodiments may determine whether breathing can be detected from the audio data. If not, then the audio data may be discarded, and the process may loop back to step 205. If yes, then the microphone (the particular second sensor, in this example) can continue gathering data for a time R_{d} (e.g., 10-60 seconds), and use the data to make a breathing rate determination in step 245. In particular embodiments, the value of R_{d} may vary based on, e.g., a user's preferences and/or on the user's current motion. For example, if a user's motion indicates the user is resting, then R_{d} may be relatively short. On the other hand, if a user's motion indicates that the user is active, or that the user is undergoing unclassified movement (e.g., “other”), then R_{d} may be relatively longer.

As an example of determining whether breathing can be detected from the audio data, particular embodiments may use a lightweight algorithm to detect breathing events. For example, the audio data may first be normalized, e.g., using min-max normalization. Then, a sliding window with, e.g., a 4 second window size and a 1 second step size may be applied to the normalized data. For the window of each step, features (e.g., a relatively low number of features such as 30 features) can be extracted and passed to a trained machine-learning classifier, such as a random forest classifier with 20 estimators (which may use only about 620 KB of storage space). The classifier detects breathing events in each window. Then, embodiments may use a clustering approach to identify the clusters of breathing audio (e.g., clusters corresponding to “inhale,” “pause”, “or” exhale) in some or all of the full breathing-data segment having a duration R_{d}. Since a typical breathing rate will include multiple breathing episodes during R_{d}, the number of detected breathing episodes can be used (e.g., by comparing the number to a threshold number) to determine whether the audio data is sufficiently robust to perform a breathing-rate determination. If yes, then step 245 may be performed. If not, then the process may return to step 205, or in particular embodiments, the process may return to step 240 to gather more data. While the example above is described in the context of audio data, i.e., the second sensor is a microphone, this disclosure contemplates that a similar process may be used to analyze data from any other second sensor.

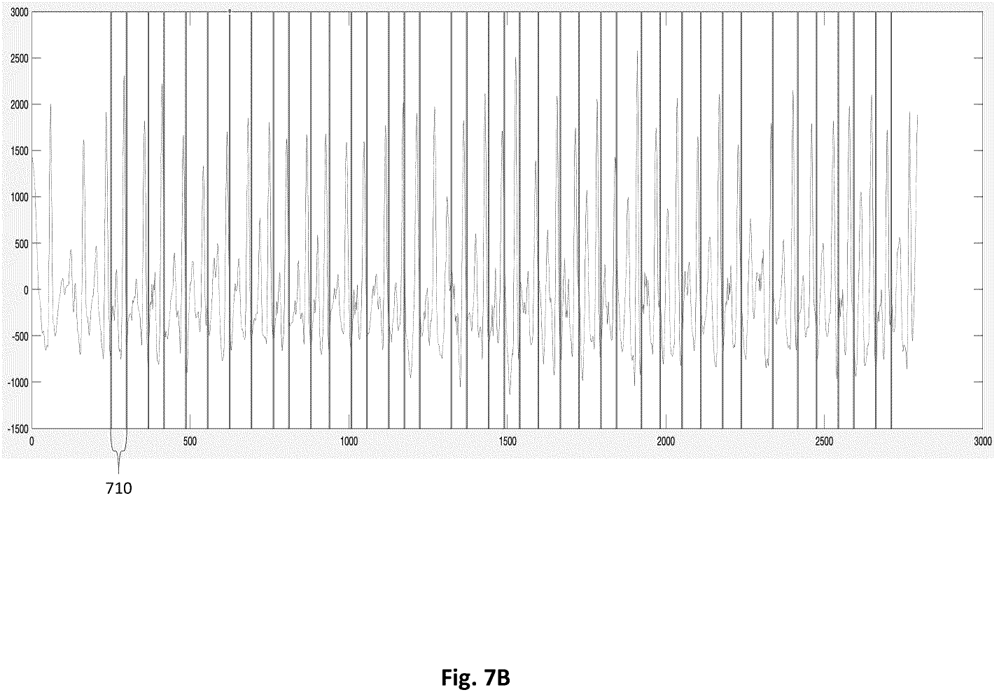

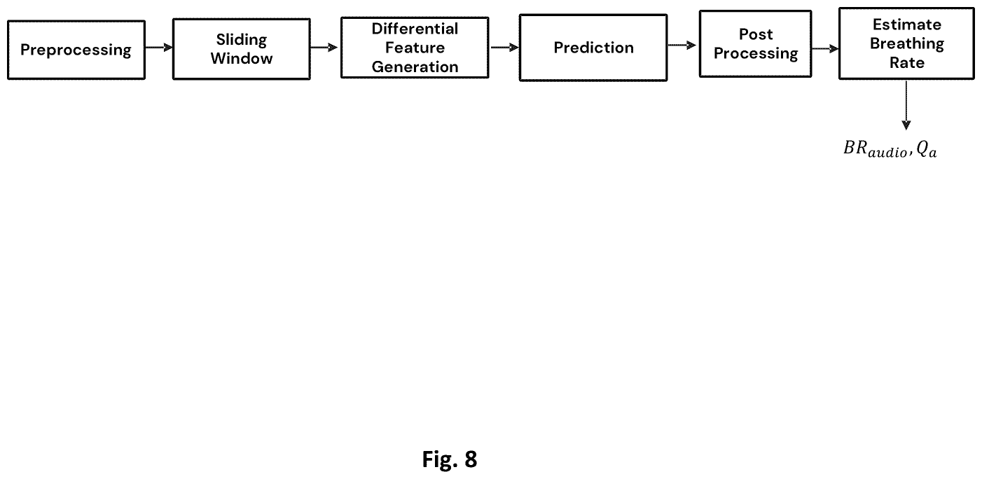

FIG. 8 illustrates an example of step 245. As explained below, the process of FIG. 8 uses a machine-learning classifier to detect transitions between breathing phases, and then uses these classifications to calculate the breathing rate. A transition is a change in breathing phase, from inhale to exhale or from exhale to inhale, for example as illustrated by transitions 910 and 915 in the example of FIG. 9. Transition 910 begins an inhale phase, and transition 915 begins an exhale phase. Particular embodiments only need to identify the start of breathing phases, or the transitions, to determine breathing rates. A transition phase always indicates a change of energy, and most of the time, the start of the breathing phase, which corresponds to the transition, has higher energy than the end of the previous breathing phase.

In the example of FIG. 8, preprocessing may include applying a window to the data and using a min-max normalization for normalizing the window. Then, a sliding window, e.g., with a 4 second window size and a 1 second step size, may be used to analyze the data. Since transitions can be instantaneous, particular embodiments may use another machine-learning classifier on a smaller window (e.g., 800 milliseconds) with a 100 millisecond step size to identify the transitions in the breathing signals.

In the example of FIG. 8, differential feature generation may include dividing the 800 ms window into two 400 ms windows, e.g., a ‘left’ window (such as window 902 in the example of FIG. 9) and a ‘right’ window (such as window 904 in the example of FIG. 9). Particular embodiments may then calculate the difference between the signals in the two windows, and these differences are the differential features. To reduce the computational overhead, particular embodiments may select only a relatively low number of features (e.g., 30) that have a high correlation with the breathing sign to use during the prediction step.

In the example of FIG. 8, prediction can include using a lightweight classifier to detect the transition points in the 800 ms window, for example because the transition prediction classifier may run every 100 ms (i.e., the classifier is outputting a prediction each time the window is stepped by, in this example, 100 ms). Particular embodiments may use a multilayer perceptron (MLP) architecture having a network size of 15×30×15, which my take only about 45 KB of storage. In particular embodiments, the classifier may use three classes: (1) breathing, (2) transition, and (3) noise. In particular embodiments, the noise class is useful at least because there can be some instantaneous noises, which can otherwise be incorrectly labeled as a transition.

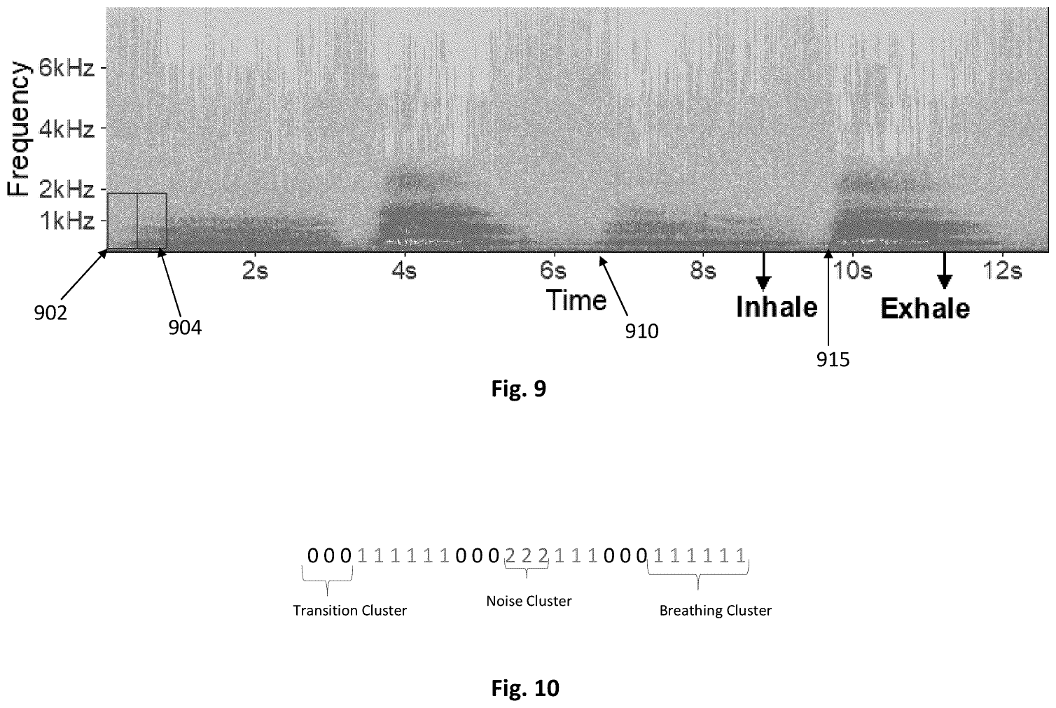

In the example of FIG. 8, post processing can include clustering the labelled classifications. For example, because particular embodiments generate a prediction every 100 ms, in such embodiments there will be small clusters of different classes. Particular embodiments use a clustering approach to identify the start and end of the clusters. FIG. 10 illustrates an example set of classifications output by the prediction step of FIG. 8 and illustrates an example of clustering such classifications. In this example, the class label “0” indicates a transition class, label “1” indicates a “breathing” class, and label “2” indicates a noise class. Particular embodiments may adjust class labels based on e.g., a presence of one or more outlier labels within a block of otherwise similarly labeled output. For example, if 4 out of 6 consecutive labels belong to a particular class and the two outliers are interspersed within the 4 similar class labels, then the two non-conforming class labels may be changed to be the particular class label, i.e., such that all 6 labels are the same. The threshold for reclassification can depend on, e.g., the type of classification label being considered.

In particular embodiments, a breathing rate estimation in the example of FIG. 8 can be based on the time between transition clusters. For example, the time between adjacent transition clusters dₜ can be calculated, and the mean or median duration between transitions can be used to determine the user's breathing rate. For example, the breathing rate can be calculated from the median transition duration as

$$B {R}_{audio} = \frac{60}{2 * median ( {d}_{t} )}$$

Particular embodiments may determine a quality associated with the breathing rate determined by the second sensor. For instance, in the example above, there should be many (e.g., 10) breathing cycles in the last 60 seconds of audio data at a normal breathing rate, which is greater than 5 breaths per minute. Since the number of clusters provides an estimate of the number of cycles, the number of the clusters (Nc, which here is the combination of the number of transitions and breathing clusters) can be used as one quality parameter. In addition or the alternative, the size of clusters Sc may be used during post processing. For example, when a 100 ms sliding window is used, there should be multiple transitions detected in a cluster, and if Sc is small for a particular cluster, that could indicate that the classifier might have detected a false transition. Therefore, particular embodiments discard all small transition clusters, e.g., clusters that that have fewer than 3 elements in the cluster, meaning at least 3 transition class labels are needed (in this example) in a cluster for valid breathing-rate determination.

In particular embodiments, a noise to breathing ratio NBR may be used to determine the quality of a breathing-rate determination. For, example, NBR may be defined as:

$$\frac{No . Of Noise Clusters Detected}{No . of Breathing Clusters Detected}$$

Since noise signals can mask the breathing signals, high NBR could mean that the breathing signals are too noisy to make accurate breathing-rate predictions. In particular embodiments, a quality Qₐ for a breathing predication may be defined as:

$${Q}_{a} = \{ \begin{matrix}0 ; if Nc < 10 and NBR > 2 \\ 1 ; else\end{matrix} \}$$

If the quality is below a threshold (which would be 1 in the example above), then the breathing-rate calculation made by the second sensor may be discarded, and the process may loop back to step 205 in some embodiments or step 240 in other embodiments. If the quality is not less than the threshold, then the determined breathing rate may be used, for example as described below. The process then returns to step 205.

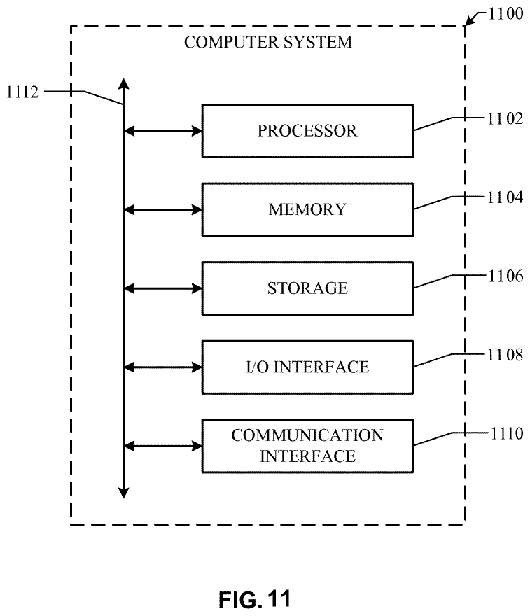

Particular embodiments may repeat one or more steps of the method of FIG. 2, where appropriate. Although this disclosure describes and illustrates particular steps of the method of FIG. 2 as occurring in a particular order, this disclosure contemplates any suitable steps of the method of FIG. 2 occurring in any suitable order. Moreover, although this disclosure describes and illustrates particular components, devices, or systems carrying out particular steps of the method of FIG. 2, such as the computer system of FIG. 11, this disclosure contemplates any suitable combination of any suitable components, devices, or systems carrying out any suitable steps of the method of FIG. 2. Moreover, this disclosure contemplates that some or all of the computing operations described herein, including the steps of the example method illustrated in FIG. 2, may be performed by circuitry of a computing device, for example the computing device of FIG. 11, by a processor coupled to non-transitory computer readable storage media, or any suitable combination thereof. In particular embodiments, some or all of the steps of the example of FIG. 2 may be performed by the wearable device, for example by electronics including components of the example computing device of FIG. 11, such as a microcontroller. In particular embodiments, some or all of the steps of the example of Fig. may be performed by one or more other devices, such as a client device (e.g., a smartphone) or a server device, or both.

Steps 205-235 may consume less power than that consumed by steps 240-245. For example, a pair of earbuds continuously executing steps 205-235 on a pair of earbuds may see a decrease in battery life of 4%-6% per hour, while when not executing those steps the battery may decrease by 3%-5% per hour. Steps 240-245 executing continuously on the earbuds when the second sensor is a microphone may decrease battery life by nearly 20%, in contrast, illustrating the efficiency of the example method of FIG. 2 that enables continuous, passive, and accurate breathing rate determinations.

In particular embodiments, one or more aspects of a breathing-rate determination may be based on user preferences and/or on user personalization. For example, parameters associated with an audio activator and/or an audio recording scheduler may be based on in part on a user's personalization. For instance, if a user's audio tends to be less useful, e.g., because the user's audio energy tends to be insufficient for capturing breathing rates, then a system may run an audio activator less frequently and/or may record audio segments for longer. In particular embodiments, if the user's audio tends to be relatively more useful, e.g., because the user's breathing rate tends is readily detectable from the audio signal, then a system may run an audio activator more frequently. As another example, particular embodiments may activate a second sensor, such as microphone, based on user preferences for, e.g., accuracy, power consumption, etc.

In particular embodiments, a frequency of activation of a second sensor can be based on a combination of factors such as a user preference, the accuracy of breathing determinations made by the second sensor, and/or the accuracy of breathing determinations made using the motion sensor. For example, a user may select among a variety of possible preferences regarding how frequently a sensor should activate to detect the user's breathing rate. For example, a user could select a “frequent” activation setting, which may correspond to, e.g., activating a microphone every one minute. As another example, a user could select a “periodic” activation setting, which may correspond to, e.g., activating a microphone every 5-10 minutes. As another example, a user could select a “sporadic” activation setting, which may correspond to, e.g., activating a microphone every 30 minutes.

In particular embodiments, a frequency of activation of a second sensor can be based at least in part on an accuracy of breathing determinations made by the motion sensor. In particular embodiments, the accuracy may be personalized for a particular user, so that if that user's motion-based breathing rate is relatively inaccurate, then a second sensor (e.g., microphone) may be activated relatively more frequently. As an example, a breathing relevance score based on an IMU data may be determined. First, motion-based data can be obtained, either during an initialization process or by accessing chunks of breathing-related motion data obtained when a user is at rest. From these recorded signals, the system can determine the most periodic axis for breathing cycle detection. The system can then perform noise filtering on the recorded signal, for example by first performing median filtering and then performing a bandpass filter and Savitzky Golay filter. The system can then use a peak-detection algorithm to determine the peaks and valleys of the signal. Each valley-to-valley or peak-to-peak segment can be labelled as a breathing cycle. The system can then select, e.g., at random, some breathing cycles to determine the quality of the breathing cycles obtained from IMU data for that user. Particular embodiments may then calculate the Dynamic Time Warping distance (DTW) for each breathing cycles with some pre-selected good breathing cycles, e.g., as collected from data from many users. The average DTW distance for each cycle is then combined to calculate the breathing quality score for the IMU-based breathing rate determinations for that user. If the DTW distance is relatively low, that means the quality score is relatively high, and a second sensor (e.g., microphone) can be activated less frequently. If the DTW distance is relatively high, then the relevance score is relatively low and a second sensor can be activated relatively more frequently.

Activation frequency of a second sensor, such as a microphone, may be based at least in part on the personalized, user-specific quality of breathing-rate determinations by the corresponding second sensor. For example, a pipeline may be used to calculate a breathing relevance score for the user, for example during an initialization phase in which samples are collected from a user breathing over a period of time (e.g., 1 minute), or by identifying a set of data segments (e.g., audio segments) in which the breathing data is discernible. For example, particular embodiments may access a series of short audio recordings taken at rest by the user, and from the recordings attempt to detect breathing phases, such as described in U.S. Patent Application Publication No. US 2022/0054039, of which the corresponding disclosure is incorporated herein. The system may identify the ‘inhale’ and ‘exhale’ phases and merge consecutive breathing phases to construct a breathing cycle. Based on the detected breathing cycles, the system can select some of the best N breathing cycles, e.g., those that have maximal breathing energy relative to the signal's total energy. The number of the best breathing cycle can be variable, and in particular embodiments, three cycles is sufficient to determine the user's breathing pattern. Once the system selects the best breathing cycles, the system can determine the probability that this particular user will provide audio data that has good breathing cycles going forward. For each of the best N breathing cycles, the system can extract audio-based features and pass these features into to a breathing-cycle classifier. The classifier yields the probability of a good breathing cycle for each of the extracted cycles, which then can be averaged to calculate a breathing quality score. A relatively high breathing relevance (quality score) indicates that an audio-based algorithm might be relatively more useful to determine that user's breathing rate, and therefore the audio-based pipeline can be selected relatively more frequently (e.g., by increasing a motion-based quality threshold) for that user.

In particular embodiments, a second sensor may be one of a number of second sensors on a wearable device. For example, a wearable device may include a microphone, a photoplethysmography (PPG) sensor, a temperature sensor, and/or other sensors that can be used to estimate a user's breathing rate. For example, a PPG sensor can capture Respiratory Sinus Arrythmia (RSA), which may be utilized to estimate breathing rate. In particular embodiments, a quality associated with one breathing rate determined using one second sensor (e.g., a microphone) may be compared with a threshold, and if the quality is below the threshold, then another second sensor (e.g., a PPG sensor) may be used to estimate a user's breathing rate. In particular embodiments, such second sensors may be ranked in order of use when a motion-based breathing-rate determination is insufficient. For example, second sensors may be ranked based on power consumption, such that the second sensor with the lowest power consumption is ranked the highest. In particular embodiments, second sensors may be ranked based on accuracy, such that the most-accurate second sensor is ranked the highest. In particular embodiments, a ranking may be based on a combination (such as a weighted combination) of a number of factors, such as power consumption, accuracy, and user relevance for that particular user. In particular embodiments, each time a sensor fails to adequately determine a breathing rate, then the system selects the next sensor in the ranked order.

Particular embodiments may use a combination of sensors to determine a user's breathing rate. Similar to the discussion above regarding ranked second sensors, groups of sensors may be ranked for use in determining a user's breathing rate. For example, data from a group of two or more sensors may be input into a machine learning model, and this data may be concatenated to calculate the breathing rate. For example, U.S. Patent Application Publication No. 2022/0054039 describes embodiments and architectures that use multimodal system for breathing phase detection, and such disclosures are incorporated herein by reference. A motion sensor and a microphone may be one sensor group, and a motion sensor and a PPG sensor may be another group, etc. In particular embodiments, if one sensor group fails to adequately estimate a user's breathing rate, then the next sensor group can be activated. As discussed above, ranking may be performed in any suitable manner, such as by power consumption, accuracy, user relevance, or any suitable combination thereof. In particular embodiments, a sensor group may be created dynamically, such as by taking the top N ranked sensors as one group, the next M ranked sensors as another group, etc.

Monitoring a user's breathing rate can be useful for many purposes. For example, embodiments disclosed herein can be used for emergency event detection by tracking critical breathing-related conditions, including medical emergencies. Abnormality of breathing rate is directly associated with medical emergencies, and particular embodiments can trigger a warning (e.g., by providing an audio or visual alert to a user, via the wearable device and/or a connected device) if an abnormality (e.g., the breathing rate is below an emergency threshold, which may be based on the user's personalized data) is detected, for example while the user is resting.

As another example, breathing rate increases before lung condition exacerbation in many medical conditions, such as asthma and COPD, and tracking breathing rate can help users intervene or treat episodes more quickly. As another example, breathing rate plays an important role during exercise as an indicator of physical effort, often more so than other physiological variables. Particular embodiments can track the breathing rate and associate that breathing rate with an estimation or prediction of a user's physical effort.

Breathing rate is an important medical parameter. Embodiments disclosed herein can provide breathing-rate information, both in real-time and over a previous time period, to a medical professional, for example to a doctor during a telehealth appointment for remote health monitoring. As another example, embodiments disclosed herein permit breathing-rate determinations and monitoring without requiring a user to visit at a medical facility. For instance, a user who had surgery may be released from the hospital when their condition allows, without needing to keep the patient in the hospital simply to monitor the user's breathing rate to ensure the user remains stable. Breathing rate and patterns are also an important biomarker for stress detection and management, and continuous breathing rate monitoring can be useful for stress detection and early intervention.

FIG. 11 illustrates an example computer system 1100. In particular embodiments, one or more computer systems 1100 perform one or more steps of one or more methods described or illustrated herein. In particular embodiments, one or more computer systems 1100 provide functionality described or illustrated herein. In particular embodiments, software running on one or more computer systems 1100 performs one or more steps of one or more methods described or illustrated herein or provides functionality described or illustrated herein. Particular embodiments include one or more portions of one or more computer systems 1100. Herein, reference to a computer system may encompass a computing device, and vice versa, where appropriate. Moreover, reference to a computer system may encompass one or more computer systems, where appropriate.

This disclosure contemplates any suitable number of computer systems 1100. This disclosure contemplates computer system 1100 taking any suitable physical form. As example and not by way of limitation, computer system 1100 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1100 may include one or more computer systems 1100; be unitary or distributed; span multiple locations; span multiple machines; span multiple data centers; or reside in a cloud, which may include one or more cloud components in one or more networks. Where appropriate, one or more computer systems 1100 may perform without substantial spatial or temporal limitation one or more steps of one or more methods described or illustrated herein. As an example and not by way of limitation, one or more computer systems 1100 may perform in real time or in batch mode one or more steps of one or more methods described or illustrated herein. One or more computer systems 1100 may perform at different times or at different locations one or more steps of one or more methods described or illustrated herein, where appropriate.

In particular embodiments, computer system 1100 includes a processor 1102, memory 1104, storage 1106, an input/output (I/O) interface 1108, a communication interface 1110, and a bus 1112. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1102 includes hardware for executing instructions, such as those making up a computer program. As an example and not by way of limitation, to execute instructions, processor 1102 may retrieve (or fetch) the instructions from an internal register, an internal cache, memory 1104, or storage 1106; decode and execute them; and then write one or more results to an internal register, an internal cache, memory 1104, or storage 1106. In particular embodiments, processor 1102 may include one or more internal caches for data, instructions, or addresses. This disclosure contemplates processor 1102 including any suitable number of any suitable internal caches, where appropriate. As an example and not by way of limitation, processor 1102 may include one or more instruction caches, one or more data caches, and one or more translation lookaside buffers (TLBs). Instructions in the instruction caches may be copies of instructions in memory 1104 or storage 1106, and the instruction caches may speed up retrieval of those instructions by processor 1102. Data in the data caches may be copies of data in memory 1104 or storage 1106 for instructions executing at processor 1102 to operate on; the results of previous instructions executed at processor 1102 for access by subsequent instructions executing at processor 1102 or for writing to memory 1104 or storage 1106; or other suitable data. The data caches may speed up read or write operations by processor 1102. The TLBs may speed up virtual-address translation for processor 1102. In particular embodiments, processor 1102 may include one or more internal registers for data, instructions, or addresses. This disclosure contemplates processor 1102 including any suitable number of any suitable internal registers, where appropriate. Where appropriate, processor 1102 may include one or more arithmetic logic units (ALUs); be a multi-core processor; or include one or more processors 1102. Although this disclosure describes and illustrates a particular processor, this disclosure contemplates any suitable processor.

In particular embodiments, memory 1104 includes main memory for storing instructions for processor 1102 to execute or data for processor 1102 to operate on. As an example and not by way of limitation, computer system 1100 may load instructions from storage 1106 or another source (such as, for example, another computer system 1100) to memory 1104. Processor 1102 may then load the instructions from memory 1104 to an internal register or internal cache. To execute the instructions, processor 1102 may retrieve the instructions from the internal register or internal cache and decode them. During or after execution of the instructions, processor 1102 may write one or more results (which may be intermediate or final results) to the internal register or internal cache. Processor 1102 may then write one or more of those results to memory 1104. In particular embodiments, processor 1102 executes only instructions in one or more internal registers or internal caches or in memory 1104 (as opposed to storage 1106 or elsewhere) and operates only on data in one or more internal registers or internal caches or in memory 1104 (as opposed to storage 1106 or elsewhere). One or more memory buses (which may each include an address bus and a data bus) may couple processor 1102 to memory 1104. Bus 1112 may include one or more memory buses, as described below. In particular embodiments, one or more memory management units (MMUs) reside between processor 1102 and memory 1104 and facilitate accesses to memory 1104 requested by processor 1102. In particular embodiments, memory 1104 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1104 may include one or more memories 1104, where appropriate. Although this disclosure describes and illustrates particular memory, this disclosure contemplates any suitable memory.

In particular embodiments, storage 1106 includes mass storage for data or instructions. As an example and not by way of limitation, storage 1106 may include a hard disk drive (HDD), a floppy disk drive, flash memory, an optical disc, a magneto-optical disc, magnetic tape, or a Universal Serial Bus (USB) drive or a combination of two or more of these. Storage 1106 may include removable or non-removable (or fixed) media, where appropriate. Storage 1106 may be internal or external to computer system 1100, where appropriate. In particular embodiments, storage 1106 is non-volatile, solid-state memory. In particular embodiments, storage 1106 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1106 taking any suitable physical form. Storage 1106 may include one or more storage control units facilitating communication between processor 1102 and storage 1106, where appropriate. Where appropriate, storage 1106 may include one or more storages 1106. Although this disclosure describes and illustrates particular storage, this disclosure contemplates any suitable storage.

In particular embodiments, I/O interface 1108 includes hardware, software, or both, providing one or more interfaces for communication between computer system 1100 and one or more I/O devices. Computer system 1100 may include one or more of these I/O devices, where appropriate. One or more of these I/O devices may enable communication between a person and computer system 1100. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1108 for them. Where appropriate, I/O interface 1108 may include one or more device or software drivers enabling processor 1102 to drive one or more of these I/O devices. I/O interface 1108 may include one or more I/O interfaces 1108, where appropriate. Although this disclosure describes and illustrates a particular I/O interface, this disclosure contemplates any suitable I/O interface.

In particular embodiments, communication interface 1110 includes hardware, software, or both providing one or more interfaces for communication (such as, for example, packet-based communication) between computer system 1100 and one or more other computer systems 1100 or one or more networks. As an example and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1110 for it. As an example and not by way of limitation, computer system 1100 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1100 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1100 may include any suitable communication interface 1110 for any of these networks, where appropriate. Communication interface 1110 may include one or more communication interfaces 1110, where appropriate. Although this disclosure describes and illustrates a particular communication interface, this disclosure contemplates any suitable communication interface.

In particular embodiments, bus 1112 includes hardware, software, or both coupling components of computer system 1100 to each other. As an example and not by way of limitation, bus 1112 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1112 may include one or more buses 1112, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. One or more non-transitory computer readable storage media storing instructions and coupled to one or more processors that are operable to execute the instructions to:
access motion data obtained by a first sensor of a wearable device from motion of a user wearing the wearable device;
determine, from the motion data, an activity level of the user;
select, based on the determined activity level, an activity-based technique for estimating the breathing rate of the user;
determine, using the selected activity-based technique and the accessed motion data, a breathing rate of the user;
determine a quality associated with the determined breathing rate;
compare the determined quality associated with the determined breathing rate with a threshold quality; and
in response to a determination that the determined quality is not less than the threshold quality, then use the determined breathing rate as a final breathing-rate determination for the user; and
in response to a determination that the determined quality is less than the threshold quality, then:
activate a second sensor of the wearable device for a period of time; and
determine, based on data from the second sensor, a breathing rate for the user.

2. The media of claim 1, wherein the wearable device comprises a head-worn device.

3. The media of claim 1, wherein the activity level is selected from a set of activity levels comprising a resting activity level and a moving activity level.

4. The media of claim 3, further coupled to one or more processors that are operable to execute the instructions to determine, using a resting activity-based technique associated with the resting activity level, the breathing rate of the user by:
applying a sliding window to the accessed data;
selecting, from the accessed motion data, motion data corresponding to a particular axis;
filtering the selected motion data corresponding to the particular axis; and
estimating, based on the filtered motion data, a breathing rate for the user.

5. The media of claim 4, further coupled to one or more processors that are operable to execute the instructions to:
generate a plurality of breathing-rate estimates from a plurality of different breathing-rate algorithms; and
estimate the breathing rate for the user by interpolating the plurality of breathing-rate estimates.

6. The media of claim 3, further coupled to one or more processors that are operable to execute the instructions to determine, using a moving activity-based technique associated with the moving activity level, the breathing rate of the user by segmenting the accessed motion data into motion segments, each motion segment corresponding to a breathing cycle of the user.

7. The media of claim 1, wherein the second sensor comprises a microphone.

8. The method of claim 7, further coupled to one or more processors that are operable to execute the instructions to determine a quality of the breathing rate determined based on data from the microphone, wherein the quality is based at least on (1) a noise-to-breathing ratio associated with the data from the microphone, and (2) a number of breathing clusters identified in the data from the microphone.

9. The method of claim 7, further coupled to one or more processors that are operable to execute the instructions to:
classify data from the microphone using a set of classes comprising a breathing class and a transition class;
cluster the classified data into a plurality of clusters; and
determine the breathing rate of the used based on a number of clusters having the transition class label.

10. The media of claim 1, further coupled to one or more processors that are operable to execute the instructions to:
determine whether any activity-based technique for estimating the breathing rate of the user corresponds to the determined activity level; and
when no activity-based technique for estimating the breathing rate of the user corresponds to the determined activity level, then activate the second sensor of the wearable device.

11. The media of claim 1, further coupled to one or more processors that are operable to execute the instructions to, in response to a determination that the determined quality is less than the threshold quality, determine whether to activate the second sensor based on one or more of:
an amount of elapsed time since the second sensor was last activated; or
an amount of elapsed time since the user's breathing rate was last validly determined.

12. The media of claim 1, wherein a value of the threshold quality depends on one or more of:
a user preference;
a user-specific breathing-quality score associated with the first sensor; or
a user-specific breathing-quality score associated with second sensor.

13. The media of claim 1, wherein the second sensor is one of a plurality of second sensors, wherein each of the plurality of second sensors is ranked based at least on that sensor's power consumption and detection accuracy.

14. The media of claim 1, further coupled to one or more processors that are operable to execute the instructions to periodically repeat the procedure of claim 1.

15. A method comprising:
accessing motion data obtained by a first sensor of a wearable device from motion of a user wearing the wearable device;
determining, from the motion data, an activity level of the user;
selecting, based on the determined activity level, an activity-based technique for estimating the breathing rate of the user;
determining, using the selected activity-based technique and the accessed motion data, a breathing rate of the user;
determining a quality associated with the determined breathing rate;
comparing the determined quality associated with the determined breathing rate with a threshold quality; and
in response to a determination that the determined quality is not less than the threshold quality, then using the determined breathing rate as a final breathing-rate determination for the user; or
in response to a determination that the determined quality is less than the threshold quality, then:
activating a second sensor of the wearable device for a period of time; and
determining, based on data from the second sensor, a breathing rate for the user.

16. The method of claim 15, wherein the wearable device comprises a head-worn device.

17. The method of claim 15, wherein the activity level is selected from a set of activity levels comprising a resting activity level and a moving activity level.

18. A system comprising:
one or more non-transitory computer readable storage media storing instructions; and one or more processors coupled to the non-transitory computer readable storage media, the one or more processors being operable to execute the instructions to:
access motion data obtained by a first sensor of a wearable device from motion of a user wearing the wearable device;
determine, from the motion data, an activity level of the user;
select, based on the determined activity level, an activity-based technique for estimating the breathing rate of the user;
determine, using the selected activity-based technique and the accessed motion data, a breathing rate of the user;
determine a quality associated with the determined breathing rate;
compare the determined quality associated with the determined breathing rate with a threshold quality; and
in response to a determination that the determined quality is not less than the threshold quality, then use the determined breathing rate as a final breathing-rate determination for the user; and
in response to a determination that the determined quality is less than the threshold quality, then:
activate a second sensor of the wearable device for a period of time; and
determine, based on data from the second sensor, a breathing rate for the user.

19. The system of claim 18, wherein the wearable device comprises a head-worn device.

20. The system of claim 18, wherein the activity level is an activity level selected from a set of activity levels comprising a resting activity level and a moving activity level.
